(19) United States
(12) Patent Application Publication
(10) Pub. No.: US 2006/0019870 A1
(43) Pub. Date: Jan. 26, 2006

(54) Apoprotein cochleate compositions

(76) Inventors: Francis E. O'Donnell JR., Town and Country, MO (US); Susan Gould-Fogerite, Annandale, NJ (US); Raphael J. Mannino, Annandale, NJ (US)

Correspondence Address: LAHIVE & COCKFIELD, LLP., 28 STATE STREET, BOSTON, MA, 02109, US

(21) Appl. No.: 11/047,373

(22) Filed: Jan. 28, 2005

Related U.S. Application Data
(60) Provisional application No. 60/540,269, filed on Jan. 28, 2004.

(51) Int. Cl. A61K 38/17; A61K 31/685

(52) U.S. Cl. 514/2; 514/78

(57) ABSTRACT

Disclosed herein are cochleates and cochleate compositions associated with an apoprotein component and/or a plasmid component that encodes an apoprotein. Also disclosed are methods of making and using the compositions of the invention, including methods of administration. Use of the invention provides safe, effective and efficient delivery of apoproteins and/or plasmids encoding the same in a variety of dosage forms.

### RELATED APPLICATIONS

This application claims priority to U.S. Provisional Patent Application Ser. No. 60/540,269, filed on Jan. 28, 2004, the entire contents of which are incorporated by this reference. This application is also related to U.S. Provisional Patent Application Serial Nos. 60/422,989, filed Nov. 1, 2002; 60/440,284, filed Jan. 14, 2003; 60/507,361, filed Sep. 29, 2003; 60/502,557, filed Sep. 11, 2003; 60/537,252, filed Jan. 15, 2004; 60/499,247,filed Aug. 28, 2003; and 60/532,755, filed Dec. 24, 2003, the entire contents of which are all incorporated herein in their entireties by this reference. This application is also related to U.S. Utility patent application Ser. No; 10/105,314, filed Mar. 26, 2002; Ser. No. 10/304,567 filed Nov. 26, 2002; Ser. No. 10/701,364, filed Nov. 3, 2003; and Ser. No. 10/822,230, filed Apr. 9, 2004

### TECHNICAL FIELD

The invention generally relates to cochleate delivery vehicles that include an apoprotein (e.g., an apolipoprotein) and/or plasmids that encode for apoproteins, and methods of manufacture and administration.

### BACKGROUND

Cochleate structures were first prepared by D. Papahadjopoulos as an intermediate in the preparation of large unilamellar vesicles (see U.S. Pat. No. 4,078,052). The use of cochleates to deliver molecules has been disclosed, e.g., in U.S. Pat. No. 5,994,318.

Methods of forming cochleates have been disclosed. For example, U.S. Pat. No. 6,153,217 describes a process for producing a cochleate precipitate having a mean particle size less than one micron. The cochleate precipitate is derived from liposomes which are suspended in an aqueous two-phase polymer solution, enabling the differential partitioning of polar molecule based-structure by phase separation. The liposome-containing two-phase polymer solution, treated with positively charged molecules, forms a cochleate precipitate of a particle size less than one micron.

### SUMMARY OF THE INVENTION

The present invention provides novel cochleates and cochleate compositions that include an apoprotein. In one aspect, the present invention provides an apoprotein-cochleate composition including a cochleate including a negatively charged lipid component and a multivalent cation component and an apoprotein component associated with the cochleate. In one embodiment, the apoprotein component includes at least one apolipoprotein. In another embodiment, the apoprotein component includes at least one apolipoprotein selected from the group consisting of apolipoprotein A (apoA-I), apolipoprotein E (apo-E), apolipoprotein-Milano (apoA-IM), apolipoprotein-Milano dimers, proapolipoprotein (proapo AI), recombinant apoA-I, apolipoprotein B, apolipoprotein A-II, apolipoprotein A-IV, apolipoprotein C-I, apolipoprotein Lp(a), apolipoprotein alleles, and apolipoprotein D. In another embodiment, the apoprotein cochleate composition further includes an anti inflammatory drug. In another embodiment, the cochleate composition has a mean particle size of less than one micron.

In another embodiment, the apoprotein component includes an apoprotein complexed with a phospholipid. In yet another embodiment, the apoprotein component includes an apoprotein bound to a cochleate component. In still another embodiment, the apoprotein is bound by an electrostatic, hydrophobic, covalent, or ionic interaction with the cochleate component. In another embodiment, the apoprotein is bound to the cochleate component with a digestible, reducible, or otherwise reversible linker. In yet another embodiment, the invention provides a composition of any of the preceding compositions further including a pharmaceutically acceptable carrier. In another embodiment, the invention provides a composition of any of the preceding compositions, wherein the composition is in the form of a pill, capsule, lozenge, or liquid for oral administration.

In yet another aspect, the present invention provides a method for making an apoprotein cochleate. This method generally includes introducing an apoprotein to a liposomal suspension or lipid solution and contacting the resulting suspension or solution with a multivalent cation to form an apoprotein cochleate.

In another aspect, the invention provides a method for treating a disease or disorder in a subject including: administering to said subject a therapeutically effective amount of a cochleate composition including a cochleate including a negatively charged lipid component and a multivalent cation component, and an apoprotein component associated with the cochleate. In one embodiment, the apoprotein component includes at least one apolipoprotein. In another embodiment, the apoprotein component includes at least one apolipoprotein selected from the group consisting of apolipoprotein A (apoA-I), apolipoprotein E (apo-E), apolipoprotein-Milano (apoA-IM), apolipoprotein-Milano dimers, proapolipoprotein (proapo AI), recombinant apoA-I, apolipoprotein B, apolipoprotein A-II, apolipoprotein A-IV, apolipoprotein C-I, apolipoprotein Lp(a), apolipoprotein alleles, and apolipoprotein D.

In yet another embodiment, the apoprotein component includes an apoprotein complexed with a phospholipid. In still another embodiment, the apoprotein component is associated with the cochleate, such that the apoprotein component dissociates with the cochleate upon contact with a target environment. In another embodiment, the apoprotein is bound to a component of the cochleate with a linker that is reducible or otherwise reversible in the extracellular or intracellular environment of the host. In another embodiment, the apoprotein is bound to a component of the cochleate with a linker that is digestible by an enzyme endogenous to the target environment. In still another embodiment, the enzyme is an extracellular enzyme (e.g. plasma or tissue enzyme), or intracellular or endosomal enzyme endogenous to the subject. In another embodiment, the apoprotein component is electrostatically associated with the cochleate and dissociates with the cochleate upon contact with a pH gradient in a cell or organ of the subject. In yet another embodiment, the cochleate composition induces reverse cholesterol transport in one or more macrophages. In another embodiment, the disease or disorder is atherosclerosis, hypertension, hyperlipidemia, hypercholesterolemia, Alzheimer's disease, coronary heart disease, or cardiovascular disease. In other embodiments, the treatment reduces the level of artherosclerotic plaque in the subject, reduces serum cholesterol levels in the subject, increases the exflux of cholesterol from macrophages in a subject, or lessens or alleviates hypercholesterolemia. In still another embodiment, the administration route is selected from the group consisting of mucosal, systemic, oral, intranasal, intraocular, intrarectal, intravaginal, intrapulmonary, intravenous, intramuscular, subcutaneous, transdermal and intradermal.

In another aspect, the invention provides a cochleate composition including a cochleate including a negatively charged lipid component and a multivalent cation component, and a plasmid that encodes for an apoprotein associated with the cochleate. In one embodiment, the plasmid encodes at least one apolipoprotein. In another embodiment, the plasmid encodes at least one apolipoprotein selected from the group consisting of apolipoprotein A (apoA-I), apolipoprotein E (apo-E), apolipoprotein-Milano (apoA-IM), apolipoprotein-Milano dimers, proapolipoprotein (proapo AI), recombinant apoA-I, apolipoprotein B, apolipoprotein A-II, apolipoprotein A-IV, apolipoprotein C-I, apolipoprotein Lp(a), apolipoprotein alleles, and apolipoprotein D.

In another embodiment, the cochleate composition including a plasmid further includes an apoprotein, e.g., the apoprotein encoded by the plasmid. In another embodiment, the cochleate composition further includes an anti inflammatory drug. In still another embodiment, the cochleate composition includes both an apoprotein and an anti inflammatory drug.

### DETAILED DESCRIPTION OF THE INVENTION

A novel approach to the delivery of apoproteins (e.g, apolipoprotein A) has now been discovered, thus providing improved modes of treatment of a number of diseases and disorders (e.g., atherosclerosis and coronary heart disease). The present invention employs cochleate delivery vehicles to protect and deliver aproproteins to a subject. By practicing the invention, the practitioner can now administer apoproteins in a variety of dosage forms (e.g., oral capsules and liquids) in a safe and effective manner.

In one embodiment, the apoprotein cochleates of the invention are employed to reduce plaque and reverse atherosclerosis in a subject. Atherosclerosis, a type of arteriosclerosis (hardening of the arteries), is the term for the build up of materials, such as fatty substances, cholesterol, cellular waste products, calcium and fibrin, in the inner lining of an artery. The resulting build up, arthrosclerotic plaque, includes dense, inflammatory agglomerations of cholesterol-laden pathologic macrophages (i.e., foam cells). Studies performed with animals indicate that hepatic overexpression of apoA-I inhibits atherosclerosis (Circulation 1996; 94: 713-717). This is believed to be due in part to the inverse association between plasma high-density lipoprotein (HDL), of which Apolipoprotein A-I (apoA-I) is a major component, and cholesterol levels and incidence of atherosclerotic cardiovascular diseases. Thus, it is believed that apoA-I cochleates of the present invention may have the ability to reduce serum cholesterol levels, in addition to the incidence of atherosclerosis.

ApoA-I plays a significant role in removing cholesterol from cells during reverse cholesterol transport (RCT) of free cholesterol from cells in peripheral tissues (e.g., macrophages in the arterial wall), to the liver for excretion in the bile. An important anti-atherosclerotic mechanism in reverse cholesterol transport is the efflux of cholesterol to apolipoprotein A-I from macrophages. Gaus et aL. FASEB J. 2004 Jan. 20. Without wishing to be bound by any particular theory, it is believed that lipid free or lipid poor apoA-I interacts with the ATP Binding Cassett-A1 (ABCA1) receptor to pick up cholesterol and phospholipids, while apoA-I in HDL interacts with the scavenger receptor B-1 (SRB-1).

In addition, other apolipoproteins, such as apoA-IV, ApoC-I and ApoE, may be employed in addition to or as a substitute for apoA-I. Accordingly, such apolipoproteins may be administered with or without apoA-I wherever administration of apoA-I is desired.

In other embodiments, apolipoprotein-cochleates are administered to a subject to treat Alzheimer's Disease (AD). Apolipoprotein E (apoE) has been found to have the ability to bind amyloid beta peptides and influence their clearance and/or deposition. For example, apoE alleles such as apoE ε2, appear to have a protective influence on the risk of late onset AD. (Petanceska et al. J Mol Neurosci. 2003; 20(3): 395-406).

It is expected that oral and intravenous preparations of apoprotein-cochleates will have improved efficacy relative to conventional apoprotein preparations. In addition, the ability of cochleates to target macrophages should significantly increase exflux of cholesterol from macrophages in a subject.

In order to more clearly and concisely describe the subject matter of the claims, the following terms are intended to provide guidance as to the meaning of specific terms used in the present specification.

The term “aggregation inhibitor,” as used herein, refers to an agent that inhibits aggregation of cochleates.

As used herein, the term “apoprotein” refers to a protein that exists as a complex between polypeptide and a second moiety of nonpolypeptide nature, the term “apoprotein” is sometimes used to refer to the molecule divested in the latter.

As used herein, the term “apolipoprotein” refers to any of various proteins that combine with a lipid to form a lipoprotein and are a constituent of chylomicrons, high-density lipoprotein, low-density lipoprotein, and very low-density lipoprotein.

As used herein, the terms “cochleate,” “lipid precipitate,” and “precipitate” are used interchangeably to refer to lipid precipitates that generally include alternating cationic and lipid bilayer sheets typically stacked and/or rolled up with little or no internal aqueous space, wherein the cationic sheet is comprised of one or more multivalent cations. Additionally, the term “encochleated” means associated with the cochleated structure, e.g., by incorporation into the cationic sheet, and/or inclusion in the lipid bilayer.

As used herein, the term “multivalent cation” refers to any compound that has at least two positive charges, including mineral cations such as calcium, barium, zinc, iron and magnesium, and cationic cargo moieties, including polyatomic cations.

As used herein, the term “negatively charged lipid” includes lipids having a head group bearing a formal negative charge in aqueous solution at an acidic or physiological pH, and also includes lipids having a zwitterionic head group.

As used herein, the term “lipid solution” refers to solutions which include a lipid and an aqueous or non-aqueous solution or solvent in which lipids are at least partially soluble. Examples of lipid solutions may include lipids dissolved or partially dissolved in solvent or aqueous or non-aqueous solutions which include one or more detergents.

“Treatment”, or “treating” as used herein, is defined as the application or administration of a therapeutic agent (e.g., apoproteins encochleated by cochleates of the invention) to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. “Treated,” as used herein, refers to the disease or disorder being cured, healed, alleviated, relieved, altered, remedied, ameliorated improved or affected. For example, certain methods of treatment of the instant invention provide for administration of Apoprotein cochleates, such that hypercholesterolemia is lessened or alleviated.

Accordingly, in one aspect, the present invention provides an apoprotein-cochleate composition that includes a cochleate generally including a negatively charged lipid component and a multivalent cation component, and an aproprotein component associated (e.g., one or more apolipoprotein) with the cochleate.

In one embodiment, the apoprotein component includes at least one apolipoprotein selected from the following group: apolipoprotein A (apoA-I), apolipoprotein E (apo-E), apolipoprotein-Milano (apoA-IM), apolipoprotein-Milano dimers, proapolipoprotein (proapo AI), and recombinant apo A-I.

In one embodiment, the apolipoprotein is an apolipoprotein, a synthetic apolipoprotein, or a synthetic apolipoprotein fragment. An exemplary synthetic apolipoprotein is described, e.g., in U.S. patent application Publication No. 20,040,186,057, filed Nov. 13, 2003, and entitled “Synthetic single domain polypeptides mimicking apolipoprotein E and methods of use.” Another example is an Apolipoprotein E synthetic peptide that binds cholesterol-rich lipoproteins and lipoprotein receptors. Nikoulin et al., “An Apoliprotein E Synthetic Peptide Targets to Lipoproteins in Plasma and Mediates Both Cellular Lipoprotein Interactions in Vitro and Acute Clearance of Cholesterol-rich Lipoproteins In Vivo,” J. Clin. Invest. 101(1) 223-234 (Jan. 1, 1998).

The apoprotein component can include one or more apoprotein complexed with a phospholipid and/or uncomplexed aproproteins.

In some embodiments, it is desirable to present the apoprotein on the surface of a cochleate so that, e.g., it can be removed from or otherwise disassociated with the cochleate. As the cochleate degrades or “unwinds” naturally in the body, what is initially in the interior of the cochleate can become exposed over time. Thus, even if the apoproteins initially present on the surface of the cochleate are inactivated or degraded by the storage environment or gastrointestinal tract, the apoprotein preserved in the interior can be exposed and delivered in an active state.

The apoprotein can be presented on the surface of the cochleate by bonding the apoprotein to a cochleate component, e.g., a phospholipid, a cholesterol or other component, such that it may be exposed on the surface of the cochleate. Accordingly, in one embodiment, the apoprotein component includes an apoprotein bound to a cochleate component. The apoprotein can be bound in a variety of ways including, not limited to, by an electrostatic, hydrophobic, covalent, or ionic interaction with the cochleate component.

In one embodiment the apoprotein is covalently bound to a component, e.g. a phospholipid and is presented and/or delivered in a bound form.

In another embodiment, the apoprotein is bound to the cochleate component in a reversible manner (e.g., with a reducible or digestible linker) or a linker susceptible to target conditions (e.g., pH, temperature, ultrasonic energy and the like). This is particularly useful as the linker can be chosen such that it is readily digestible, e.g., by an enzyme, in the body generally or even in a target structure. Thus, e.g., a linker can be chosen such that it is degiaded by an enzyme in the plasma, interstitial fluids, in a cell (e.g. a macrophage) or in an endosome, such that it become detachable and available in unbound form in these structures. In another embodiment, the reversible linker can be an electrostatic or other bond that is broken by a change in pH, e.g., in an organ or other structure in which the cochleate experiences a pH gradient. In another embodiment, the linker is reversed by a change in temperature, e.g., by exposure to body temperature.

In another aspect, the present invention provides a cochleate composition that includes a cochleate including a negatively charged lipid component and a multivalent cation component, and a plasmid that encodes for an apoprotein (e.g., an apolipoprotein) associated with the cochleate.

In one embodiment, the plasmid encodes at least one apolipoprotein selected from the group consisting of apolipoprotein A (apoA-I), apolipoprotein E (apo-E), apolipoprotein-Milano (apoA-IM), apolipoprotein-Milano dimers, proapolipoprotein (proapo AI), and recombinant apoA-I.

In another embodiment, the cochleate composition including a plasmid further includes an apoprotein, e.g., the apoprotein encoded by the plasmid. In another embodiment, the cochleate composition further includes an anti inflammatory drug, In still another embodiment, the cochleate composition includes both an apoprotein and an anti inflammatory drug.

Because cochleates are stable structures which can withstand a variety of physiologic conditions, cochleates are suitable means for delivering biologic molecules, such as plasmids polynucleotides, to a selected site in a host.

The plasmid or polynucleotide is generally one which expresses an apoprotein, such as the apoproteins described herein.

The cochleates of the invention provide protection to encochleated apoproteins and/or plasmids from, for example, digestion in the stomach. The cochleate structure provides protection from degradation for associated “encochleated” molecules. Divalent cation concentrations in vivo in serum and mucosal secretions are such that the cochleate structure is maintained. Since the cochleate structure includes a series of solid layers, components within the interior of the cochleate structure remain substantially intact, even though the outer layers of the cochleate may be exposed to harsh environmental conditions or enzymes.

In addition, the cochleate interior not only is primarily free of water, but also is resistant to penetration by oxygen. Oxygen and water are prirmaily responsible for the decomposition and degradation of associated components which leads to reduced shelf-life. Accordingly, encochleation also imparts extensive shelf-life stability to the apoproteins and/or plasmids.

With respect to storage, cochleates can be stored in cation-containing buffer, e.g., a moderately or highly concentrated suspension/solution of cochleates, or lyophilized or otherwise dried to a powder, and stored at room temperature. If desired, the cochleates also can be reconstituted with liquid prior to administration. Cochleate preparations have been shown to be stable for more than two years at 4° C. in a cation-containing buffer, and at least one year as a lyophilized powder at room temperature.

The lipid employed in the present invention preferably includes one or more negatively charged lipids. In one embodiment, the lipid is a mixture of lipids, comprising at least 75% negatively charged lipid. In another embodiment, the lipid includes at least 85% negatively charged lipid. In other embodiments, the lipid includes at least 90%, 95% or even 99% negatively charged lipid. All ranges and values between 60% and 100% negatively charged lipid are meant to be encompassed herein.

The negatively charged lipid can include soy-based lipids. Preferably, the lipid includes phospholipids, such as soy-based phospholipids. The negatively charged lipid can include phosphatidylserine (PS), phosphatidic acid (PA), phosphatidylinositol (PI), and/or phosphatidyl glycerol (PG) and/or a mixture of one or more of these lipids with other lipids. Additionally or alternatively, the lipid can include phosphatidylcholine (PC), phosphatidylethanolamine (PE), diphosphotidylglycerol (DPG), and the like. Fatty acids may also be included.

The lipids can be natural or synthetic. For example, the lipid can include esterified fatty acid acyl chains, or organic chains attached by non-ester linkages such as ether linkages (as described in U.S. Pat. No. 5,956,159), disulfide linkages, and their analogs.

In one embodiment the lipid chains are from about 6 to about 26 carbon atoms, and the lipid chains can be saturated or unsaturated. Fatty acyl lipid chains useful in the present invention include, but are not limited to, n-tetradecanoic, n-hexadecanoic acid, n-octadecanoic acid, n-eicosanoic acid, n-docosanoic acid, n-tetracosanoic acid, n-hexacosanoic acid, cis-9-hexadecenoic acid, cis-9-octadecenoic acid, cis,cis-9,12-octadecedienoic acid, all-cis-9,12,15-ocatecetrienoic acid, all cis-5,8,ll,14-eicosatetraenoic acid, all-cis-4,7,10,13,16,19-docosahexaenoic acid, 2,4,6,8-tetramethyl decanoic acid, and lactobacillic acid, and the like.

In some embodiments, pegylated lipid also is included. Pegylated lipid includes lipids covalently linked to polymers of polyethylene glycol (PEG). PEG's are conventionally classified by their molecular weight, thus PEG 6,000 MW, e.g., has a molecular weight of about 6000. Adding pegylated lipid generally will result in an increase of the amount of compound (e.g., peptide, nucleotide, and nutrient) that can be incorporated into the precipitate. An exemplary pegylated lipid is dipalmitoylphosphatidylehtanolamine (DPPE) bearing PEG 5,000 MW.

In yet another aspect, the present invention provides a geodate delivery vehicle which includes an apoprotein. The apoprotein may be any of the apoproteins described herein.

The term “geodate delivery vehicle” refers to a delivery vehicle for a cargo moiety. Geodate delivery vehicles are generally described in WO 04/041247, the entire contents of which are incorporated herein by this reference. Geodate delivery vehicles generally include a lipid monolayer disposed about a hydrophobic domain. A “hydrophobic domain” is a composition that is sufficiently hydrophobic in nature to allow formation of a lipid monolayer about its periphery. A hydrophobic domain can itself be one or more cargo moieties, or it can include a hydrophobic composition, such as oil or fat, associated with the cargo moiety, which can be, e.g., a hydrophobic or amphiphilic agent.

The geodate delivery vehicle may include a lipid monolayer disposed about a hydrophobic domain. The geodate delivery vehicle may also include a lipid strata disposed about the lipid monolayer. The lipid monolayer may include at least one phospholipid. The geodate delivery vehicle may be suspended in an aqueous environment, e.g., an emulsion, or in powder form. The geodate delivery vehicle may feature a lipid strata disposed about a hydrophobic domain without a lipid monolayer.

The term “lipid monolayer” generally refers to a lipid-containing layer one molecule thick (as contrasted with lipid bilayers that are two molecules thick). A lipid monolayer can contain further elements, such as cholesterol, steroids, or proteins. In contrast, “liposomes” refer to vesicles defined by lipid bilayers (two molecules thick) in a unilamellar or multilamellar structure.

In one aspect of the invention, the lipid monolayer includes and/or is composed primarily of negatively charged lipids. When a lipid strata is formed, the multivalent cation forms a cationic bridge between the negatively-charged lipid in the monolayer and the negatively charged lipid in the liposomes. In another embodiment, the lipid monolayer is composed primarily of positively charged lipids. In this case, the head groups interact with negatively charged lipid in the strata. In yet another embodiment, the lipid monolayer is composed primarily of neutral lipids. The coated hydrophobic domain, in this embodiment is trapped within the lipid strata, but does not ionically interact with the strata.

The term “lipid strata” refers to a structure of alternating cationic and lipid sheet-like layers. A lipid strata can be formed by introducing a cation to an emulsion containing liposomes. The lipid strata not only locks the hydrophobic domain within the geodate lipid monolayer, but can itself be associated with a cargo moiety (e.g., a hydrophilic agent disposed within the lipid strata). In one embodiment, the lipid strata entraps a hydrophobic domain. In another, the lipid strata entraps a hydrophobic domain disposed within a lipid monolayer. The cation preferably is a multivalent cation. The cation can be a divalent cation, such as Ca⁺⁺, Zn⁺⁺, Ba⁺⁺, and Mg⁺⁺. The cation can also be a multivalent cargo moiety.

One advantage geodate delivery vehicles is that apoproteins may be able to be incorporated into the vehicle at high concentrations. Another advantage is the ability to incorporate multiple apoproteins, or combinations of apoproteins and plasmids and/or cargo moieties into one geodate delivery vehicle. Incorporation into a geodate delivery vehicle is also advantageous because it provides the apoprotein with protection from both the environment, e.g., water and oxygen, and also the stomach. Additionally, the geodate delivery vehicle protects stomach from the apoprotein and/or cargo moiety. Geodate delivery vehicles are also advantageous because they can be formulated without solvent, they are highly stable, e.g., they can withstand extreme temperature and pressure, and they can mask the taste and/or odor of apoproteins and/or cargo moieties.

The hydrophobic domain is a hydrophobic composition that can be a carrier for one or more apoproteins, or the apoprotein itself. That is, the hydrophobic domain can be a hydrophobic carrier (e.g., olive oil or soy oil) associated with an apoprotein. Alternatively the hydrophobic domain can be the apoprotein or one or more cargo moieties that act as a carrier for the apoprotein.

In one embodiment, the hydrophobic domain is present in a range of between about 1% and 99%, preferably between about 1% and about 75%, more preferably between about 10%.

Accordingly, the compositions of the invention may include apoproteins and/or cargo moieties present in or associated with the hydrophobic domain, the lipid monolayer, the lipid strata, a stable emulsion (e.g., in liposomes or aqueous media), or any combination thereof. In addition, several layers of precipitate can be formed about or encrusted about the geodate delivery vehicles, with one or more apoproteins and/or cargo moieties associated therewith. Accordingly, the invention may be employed for combination drug therapy, (e.g anti-inflammatory) and/or consecutive or simultaneous release profiles, e.g., pulsed or extended release.

The present invention also provides a method of manufacturing a geodate delivery vehicle for an apoprotein cargo moiety. The method, which may be found in WO 04/041247, the entire contents of which are incorporated herein by this reference, generally includes the step of: mixing a lipid, an aqueous solution and a hydrophobic material, such that a geodate delivery vehicle is formed, which includes a lipid monolayer disposed about a hydrophobic domain. The methods of the invention can include the step of adding a cation to the emulsion to form a lipid strata about a geodate delivery vehicle. The lipid strata can be maintained in the emulsion.

An alternate method of forming a geodate delivery vehicle includes mixing a lipid and a hydrophobic material, e.g., by kneading, such that one or more geodate delivery vehicles are formed. This method can be advantageous, for example, when an aqueous environment is not desired.

The cochleates and cochleate compositions of the present invention optionally may include at least one aggregation inhibitor. The aggregation inhibitor typically is present at least on the surface of the cochleate, and may only be present on the surface of the cochleate (e.g., when the aggregation inhibitor is introduced after cochleate formation). Aggregation inhibitors can be added before, after, and/or during cochleate formation.

Aggregation inhibitors work in part by modifying the surface characteristics of the cochleates such that aggregation is inhibited. Aggregation can be inhibited, for example, by steric bulk and/or a change in the nature of the cochleate structure, e.g., a change in the surface hydrophobicity and/or surface charge. In some embodiments, aggregated cochleates may be disaggregated using alternative disaggregation method, e.g., homogenization or emulsification, and an aggregation inhibitor can be introduced in order to prevent reaggregation.

As discussed above, cochleates can be formed by the calcium induced restructuring and fusion of lipid, e.g., phospholipid such as phosphatidylserine (PS). Due to the hydrophobic nature of the surfaces of cochleates in aqueous, calcium containing solutions, cochleates formed without the aggregation inhibitors of the invention can aggregate and form larger masses, e.g., needle-like structures. Restricting and/or inhibiting the interaction of liposomes that can coalesce into cochleates at, or after the time of cation addition limits the size of the resultant cochleate crystal, and prevents aggregation into larger particles. The addition of an aggregation inhibitor (e.g., casein) to liposomes prior to the addition of calcium results in stable non-aggregated nanocochleate structures.

The type and/or amount of aggregation inhibitor used can also determine the size of resulting cochleate. The presence of an aggregation inhibitor in differing concentrations also allows regulation of cochleate size distribution.

Suitable aggregation inhibitors that can be employed in accordance with the present invention, include but are not limited to at least one of the following: povidone, casein, ε-casein, milk, albumen, serum albumen, bovine serum albumen, and rabbit serum albumen methylcellulose, ethylcellulose, propylcellulose, hydroxycellulose, hydroxymethyl cellulose, hydroxyethyl cellulose, hydroxypropyl cellulose, hydroxypropylmethyl cellulose, polyvinyl pyrrolidone, carboxymethyl cellulose, carboxyethyl cellulose, pullulan, polyvinyl alcohol, sodium alginate, polyethylene glycol, polyethylene oxide, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, carrageenan, carnauba wax, shellac, latex polymers, albumin, milk protein isolate, soy protein isolate, whey protein isolate and mixtures thereof.

A suitable aggregation inhibitor is casein. Casein is a highly phosphorylated, calcium binding protein. Without wishing to be bound to any particular theory, it is believed that calcium mediates an interaction between negatively charged lipid (e.g., PS) and casein, thereby changing the surface properties of cochleates such that aggregation is inhibited. Another suitable aggregation inhibitor is milk and other milk products such as Half and Half, cream ect. Preferred milk products also contain casein. Another suitable aggregation inhibitor is methylcellulose, carboxymethylcellulose, and related compounds. Yet another suitable aggregation inhibitor is albumen from various sources, e.g., serum albumen, bovine serum albumen, and rabbit serum albumen.

More than one aggregation inhibitor may be employed in the compositions of the invention. For example, both casein and methylcellulose may be used as an aggregation inhibitor.

In one embodiment, the cochleate compositions of the invention include an aggregation inhibitor to lipid ratio of between about 0.1:1 to about 4:1 by weight. In some embodiments, the aggregation inhibitor to lipid ratio is about 1:1. A person of ordinary skill in the art will readily be able to determine the amount of aggregation inhibitor needed to form cochleates of the desired size with no more than routine experimentation.

The cochleates of the present invention optionally may include an additional cargo moiety. A “cargo moiety” is a moiety to be encochleated, and generally does not refer to the lipid and ion employed to precipitate the cochleate. A cargo moiety may be organic or inorganic, a monomer or a polymer, endogenous to a host organism or not, naturally occurring or synthesized in vitro and the like.

Thus, examples include vitamins, minerals, nutrients, micronutrients, amino acids, toxins, microbicides, microbistats, co-factors, enzymes, polypeptides, polypeptide aggregates, polynucleotides, lipids, carbohydrates, nucleotides, starches, pigments, fatty acids, monounsaturated fatty acids, polyunsaturated fatty acids, saturated fatty acids, flavorings, essential oils, extracts, hormones, cytokines, viruses, organelles, steroids and other multi-ring structures, saccharides, metals, medicaments, proteins, marker compounds, metabolic poisons, imaging agents, antigens, porphyrins, tetrapyrrolic pigments, drugs and the like. Examples of suitable cargo moieties may be found, e.g., in WO 04/091578, the entire contents of which are incorporated herein by this reference.

Accordingly, in one embodiment, the cochleate preparation includes a combination therapeutic that targets delivery to macrophages of an apoA-I and of an anti-inflammatory, as a particularly effective way to promote plaque volume reduction by enhancing reverse cholesterol transport and by suppressing subintimal inflammatory stimuli to cholesterol accumulation. The anti-inflammatory may be separate encochleated, encochleated with the apoA-I, or added to a composition unencochleated.

Additional pharmacologically active agents may be delivered in combination with the primary active agents, e.g., the cochleates of this invention. In one embodiment, such agents include, but are not limited to agents that reduce the risk of atherosclerotic events and/or complications thereof. Such agents include, but are not limited to beta blockers, beta blockers and thiazide diuretic combinations, cholesterol-altering or cholesterol-lowering compounds, lipid-lowering or lipid-altering compounds, compounds which increase uptake of cholesterol by the liver and/or block endogenous cholesterol production, such as HMG CoA reductase inhibitors, compositions which prevent uptake of dietary cholesterol, and combinations of any of these which are effective to lower blood cholesterol levels, statins, aspirin, ace inhibitors, ace receptor inhibitors (ARBs), and the like.

Suitable beta blockers include, but are not limited to cardioselective (selective beta 1 blockers), e.g., acebutolol (e.g., Sectral™), atenolol (e.g., Tenormin™), betaxolol (e.g., Kerlone™), bisoprolol (e.g., Zebeta™), metoprolol (e.g., Lopressor™), and the like. Suitable non-selective blockers (block beta 1 and beta 2 equally) include, but are not limited to carteolol (e.g., Cartrol™), nadolol (e.g., Corgard™), penbutolol (e.g., Levatol™), pindolol (e.g., Visken™), propranolol (e.g, Inderal™), timolol (e.g., Blockadren™), labetalol (e.g., Normodyne™, Trandate™), and the like.

Suitable beta blocker thiazide diuretic combinations include, but are not limited to Lopressor HCT, ZIAC, Tenoretic, Corzide, Timolide, Inderal LA 40/25, Inderide, Normozide, and the like.

Suitable statins include, but are not limited to pravastatin (e.g., Pravachol™), simvastatin (e.g., Zocor™), lovastatin (e.g., Mevacor™), and the like.

Suitable ace inhibitors include, but are not limited to captopril (e.g., Capoten™), benazepril (e.g., Lotensin™), enalapril (e.g., Vasotec™), fosinopril (e.g., Monopril™), lisinopril (e.g., Prinivil™ or Zestril™), quinapril (e.g., Accupril™), ramipril (e.g., Altace™), imidapril, perindopril erbumine (e.g, Aceon™), trandolapril (e.g, Mavik™), and the like. Suitable ARBS (Ace Receptor Blockers) include but are not limited to losartan (e.g., Cozaar™), irbesartan (e.g., Avapro™), candesartan (e.g., Atacand™), valsartan (e.g., Diovan™), and the like.

Suitable HMG CoA reductase inhibitors that are useful in accordance with the methods and compositions of the invention are statin molecules. These include: Lovastatin (e.g., Mevacor™), Pravastatin (e.g., Pravachol™), Simvastatin (e.g., Zocor™), Fluvastatin (e.g., Lescol™), Atorvastatin (e.g., Lipitor™), or Cerivastatin (e.g, Baycol™).

Other agents that may be administered in conduction with the cochleates of the invention for treatment of atherosclerotic events and/or complications thereof are phytosterols, phytostanols and their derivatives and isomers; soy protein; soluble fibers, e.g beta-glucan from, for example, oat and psyllium, nuts, rice bran oil, each of which is particularly suitable for use in food, dietary supplements and food additive compositions. Phytosterols may be solid (e.g., powder, granules) or liquid (e.g., oil) form.

It will be obvious to a person of skill in the art that the choice of the agent for treatment of atherosclerotic events and/or complications thereof depends on the intended delivery vehicle (e.g., food, supplement, pharmaceutical) and the mode of administration. In further embodiments, the apoprotien cochleates of the present invention can be coadministered with anti-thrombotic drugs, including anti-coagulants and anti platelet drugs. Anti-coagulants, e.g., unfractionated heparins, low molecular weight heparins, pentasaccharides derived from heparin, and coumarins (vitamin K antagonists which hinder the formation/action of anticoagulant proteins such as prothrombin, factor VII, factor IX, factor X, protein C and protein S), generally inhibit generation of thrombin and formation of fibrin. Anti platelet drugs, e.g., GPIIb/IIIa integrin antagonists and aspirin, inhibit the aggregation of platelets. In still further embodiments, the apoprotien cochleates of the present invention can be coadministered with thrombolytic agents which such as tissue plasminogen activator (tPA) is a thrombolytic agent, which may dissolve blood clots.

Methods of Forming Apoprotien-Cochleates

Any known method can be used to form cochleates, including but not limited to those described in U.S. Pat. Nos. 5,994,318 and 6,153,217 and WO 04/091578, the entire disclosures of which are incorporated herein by this reference.

Briefly, one suitable procedure for making cochleates of the present invention is one wherein a lipid, e.g., a negatively charged lipid is utilized to produce a suspension of multilamellar lipid vesicles containing or surrounded by an apoprotein and/or a palsmid encoding an apoprotein, which are converted to small unilamelllar protein lipid vesicles by sonication under nitrogen or extrusion through filters or under pressure through an aperature in a mechanical device. Alternatively, to avoid damage, the apoprotein and/or plasmid can be added to the solution following sonication or extrusion. The vesicles are dialyzed at room temperature against buffered multivalent or divalent cation,( e.g., calcium chloride), or multivalent cations or polycations are added directly, resulting in the formation of an insoluble precipitate.

An additional method for preparing cochleates involves addition of apoprotein to lipid plus detergent sufficient to produce a solution. The solution next is dialyzed against buffer (e.g., 2 mM TES, 2 mM L-histidine, 100 mM NaCl, pH 7.4) to form small liposomes containing the apoprotein. A multi or polyvalent cation then is added either directly or by dialysis to form a precipitate. Alternatively, the lipid, Apoprotein, detergent solution is dialyzed against a solution containing multivalent cations, or this cation-containing solution is added directly. In either case a precipitate containing apoproteins is formed.

Another method of forming the cochleates of the present invention generally includes introducing an apoprotein to a liposome in the presence of a solvent such that the apoprotein associates with the liposome, and precipitating the liposome to form an apoprotein-cochleate.

The step of introducing an apoprotein to a liposome can be achieved in a variety of ways, all of which are encompassed within the scope of the present invention. In one embodiment, the apoprotein is introduced by introducing a solution of the solvent and the apoprotein, or an aqueous solution containing the apoprotein to the liposome. In one embodiment, the liposome is in a liposomal suspension, e.g., an aqueous liposomal suspension. In another embodiment, the solution is introduced to the liposome by dropwise addition of the solution. In other embodiments, the solution can be added by continuous flow or as a bolus. In addition the solution may be introduced to dried lipid, optionally with water added before, after or with the solution.

In another embodiment, when solvent is employed, the apoprotein is introduced to the liposome prior to or after the solvent. For example, the apoprotein may be introduced to a liposomal suspension that includes the solvent. The mixture can then be agitated, mixed, vortexed or the like to facilitate association of the apoprotein with the liposome. The apoprotein introduced may be in a powder or a liquid form.

In still another embodiment, a detergent may be added to the solution containing the apoprotein, before, after, or during the addition of the solution to the liposomes. Without wishing to be bound by any particular theory, it is believed that the addition of a detergent to the solution may facilitate the dissolution of the apoprotein. Suitable detergents include cholate, deoxycholate, octylglucoside, heterogeneous polyoxyethylene detergents such as Tween, BRIG or Triton, and other non-ionic detergents. Detergents may be employed with high salt concentration, e.g., from about 1M to about 6M, or low salt concentration, e.g., less than about 1M. In other embodiments, the detergent may be employed with a physiological salt concentration. A skilled artisan would be able to determine an appropriate detergent in a suitable amount with the appropriate salt concentration needed, e.g., to facilitate dissolution of the apoprotein, using no more than routine experimentation.

The liposome may be prepared by any known method of preparing liposomes. Thus, the liposomes may be prepared for example by solvent injection, lipid hydration, reverse evaporation, freeze drying by repeated freezing and thawing. The liposomes may be multilamellar or unilamellar, including small unilamellar vesicles (SUV). The concentration of lipid in these liposomal suspensions can be from about 0.1 mg/ml to 500 mg/ml. Preferably, the concentration of lipid is from about 0.5 mg/ml to about 50 mg/ml, more preferably from about 1 mg/ml to about 25 mg/ml.

The liposomes may be large unilamellar vesicles (LUV), stable plurilamellar vesicles (SPLV) or oligolamellar vesicles (OLV) prepared, e.g., by detergent removal using dialysis, column chromatography, bio beads SM-2, by reverse phase evaporation (REV), or by formation of intermediate size unilamellar vesicles by high pressure extrusion. Methods in Biochemical Analysis, 33:337 (1988). Liposomes made by all these and other methods known in the art can be used in practicing this invention.

In one embodiment, at least a majority of the liposomes are unilamellar. The method can further include the step of filtering a liposomal suspension and/or mechanically extruding the suspension through a small aperture that includes both MLV and ULV liposomes, such that a majority of the liposomes are ULV. In some embodiments, at least 70%, 80%, 90% or 95% of the liposomes are ULV.

The method is not limited by the method of forming cochleates. Any known method can be used to form cochleates from the liposomes of the invention (i.e., the liposomes associated with the apoprotein). In a preferred embodiment, the cochleate is formed by precipitation. The liposome can be precipitated with a multivalent cation to form an apoprotein-cochleate. An aggregation inhibitor may optionally be employed to prevent aggregation or reaggregation or to reverse aggregation. Optionally, cochleates may also be disaggregated using a disaggregation method, e.g., extrusion, homogenization or emulsification.

Any suitable solvent can be employed in connection with the present invention. Solvents suitable for a given application can be readily identified by a person of skill in the art. Preferably, the solvent is an FDA acceptable solvent. The solvent can be an organic solvent or an inorganic solvent. In one embodiment, the solvent is a water miscible solvent. Suitable solvents include but are not limited to dimethylsulfoxide (DMSO), a methylpyrrolidone, N-methylpyrrolidone (NMP), acetonitrile, alcohols, e.g, ethanol (EtOH), dimethylformamide (DMF), tetrahydrofuran (THF), and combinations thereof. In general, the apoprotein concentration within the solvent is between about 0.01 mg/ml and 200 mg/ml. Preferably, the apoprotein concentration is between about 0.05 mg/ml and about 100 mg/ml, more preferably between about 0.1 mg/ml and 20 mg/ml.

The solvent can optionally be removed at the liposome stage and/or after the cochleates are formed. Any known solvent removal method can be employed. For example, solvent may be removed from the liposomal suspension by tangential flow and/or filtration and/or dialysis, or from the cochleates by washing, filtration, centrifugation, and/or dialysis. The cochleates can be washed, e.g., with buffer or water, optimally with calcium or another cation. If employed, the detergent may also be removed using similar methods.

In another embodiment, the cochleates of the present invention are formed according to a method that generally includes the steps of: dissolving a lipid component and an apoprotein component in an organic solvent (e.g., THF) to form a solution; forming apoprotein liposomes; and exposing the liposomes to cation to form apoprotein cochleates.

The solvent or solvents selected preferably are organic solvents. Preferably, the solvent is an FDA acceptable solvent. Examples of suitable solvents include, but are not limited to tetrahydrofuran, chloroform, dichloromethane, carbon tetrachloride, butanol, hexane, ethanol, toluene, benzene, ether, petrol ether, oil or combinations thereof. THF is particularly advantageous because it is safer than conventional solvents used to form cochleates and lipsomes such as chloroform. In addition, mixtures of solvents can be employed in accordance with the present invention. Solvent mixtures can be useful, for example, for when the lipid is more readily soluble in one solvent and the apoprotein is more readily soluble in another; the solvents can be mixed before or after solubilizing the lipid and apoprotein in each.

The solvent can optionally be removed prior to the formation of liposomes and/or after the liposomes are formed. Any known solvent removal method can be employed. For example, solvent may be removed from the liposomal suspension by tangential flow and/or filtration and/or dialysis, or from the lipid-apoprotein solution by drying under a stream of nitrogen to form a film. Removal of the solvent may be advantageous because the solvent creates a favorable environment in which the apoprotein or lipid resides. Removing the favorable environment would facilitate the incorporation of the apoprotein into the cochleate structure.

Apoprotein liposomes can be formed by adding the lipid-apoprotein solution to an aqueous solution. Additionally or alternatively, the lipid-apoprotein can be agitated with an aqueous solution in order to form liposomes. The aqueous solution is preferably salt water. Salt water is highly polar, and creates an unfriendly environment for the apoprotein. The apoprotein, therefore, would be forced into the cochleate structure.

The method is not limited by the method of forming cochleates. Any known method can be used to form cochleates from the liposomes of the invention (i.e., the liposomes associated with the apoproteins). In a preferred embodiment, the cochleate is formed by precipitation. The cochleates also could be formed, e.g., by dialysis against buffered cation or any other known method. The liposome can be precipitated with a multivalent cation to form an apoprotein-cochleate.

Utilizing the methods of the invention a wide range of lipid to apoprotein ratios can be achieved. Different ratios can have varying biological activity. All ratios disclosed herein are w/w, unless otherwise indicated. In one embodiment, the ratio of lipid to apoprotein is between about 10,000:1 and 1000:1. Ratios in this range may be suitable when it is desired to administer small amounts of the moiety, (e.g., in the case of administration of radioactive agents or highly active, rare or expensive molecules). In another embodiment, the ratio is between about 8,000:1 and 4,000:1, e.g., about 6,000:1. In yet another embodiment, the ratio is between about 5,000:1 and 50:1. In yet another embodiment, the ratio of the lipid to the apoprotein is between about 20:1 and about 0.5:1. In another embodiment, the ratio of the lipid to the apoprotein is between about 1 :1 and about 10:1. In yet another embodiment, the ratio of lipid to the apoprotein is about 2:1, about 3:1, or between about 1.5:1 and 3.5:1. All individual values and ranges between about 0.25:1 and about 40,000:1 are within the scope of the invention. Further values also are within the scope of the invention.

In certain embodiments, ionic conditions can be created or adjusted to affect the efficiency of the association and/or the encochleation of the apoprotein and/or other cargo moieties. For example, increasing the salt concentration in a liposomal suspension can render the environment less hospitable to a hydrophobic or amphipathic molecule, thereby increasing liposome and cochleate loading efficiency. Ionic conditions can also affect the ultimate structure of the precipitate generated. High loads of an apoprotein can also affect the highly ordered structure observed in cochleates formed, e.g., exclusively from calcium and PS. Additionally or alternatively, pH conditions can be created or adjusted to affect the loading and structure of the resulting precipitates.

In one embodiment, the apoprotein is first stripped of any complexed lipids prior to encochleation. In another embodiment, the apoprotein is bound to a component of the bilayer of the cochleate, e.g., a phospholipid or other lipid. A peptide can be covalently bound to a lipid according to numerous known means such as, for example, by cross-linking to glycosphingolipids (Heath, T. D., et al. B.B.A. 640:66-81 (1981)); or via N-(p-aminophenyl)sterylamide (Snyder, S. L. and Vannier, W. E. B.B.A. 772:288-294 (1984)); or via the N-hydroxysuccinimide ester of palmitic acid (Huang, A. etal. J.B.C. 255:8015-8018 (1980)).

Covalently binding the peptide to the lipid by cross-linking is preferred and can be accomplished by methods well known in the art, for example, by a method using N-succinimidyl-4-(p-maleimidophenyl)butyrate (Martin, F. J., and Papahadjopoulos, D. J. Bio. Chem. 257:286-288 (1982)); N-hydroxysuccinimidyl 3-(2-pyri-dyldithio) propionate (Barbet, J. et al. J. Supra. Struct. and Cell Biochem. 16:243-258 (1981)); m-male-imidobenzoyl-N-hydroxysuccinimide ester (Hashimoto, Y. et al. J. Immuno. Methods 62:155-162 (1983)); citra-conylation (Jansons, V. K. and Mallett, P. L. Anal. Biochem. 111:54-59 (1981)), and N-succinimidyl 3-[2-pryidyldithio]-propionamido) (Carlsson, J. et al. Biochem. J. 172, 723-737).

In a preferred embodiment, the covalent bond is reversible so that the apoprotein can be detached from the cochleate under suitable conditions. For example, an apoprotein can be attached to a phospholipid via a linker that can be cleaved by an enzyme endogenous to a target tissue, organ, or structure (e.g., a plasma protein, interstitial protein, an endosome or the intracellular milieu), such that the apoprotein is delivered to the target tissue, organ or other structure. In alternative embodiments the apoprotein can be attached by any other means, for example, by electrostatic interactions and/or hydrophobic interactions.

An additional cargo moiety (including additional apoproteins) may also be employed in the methods of the present invention. It can be introduced with the apoprotein or with the liposome. Preferably, it is incorporated into the liposomal suspension or a solution of the apoprotein and solvent.

Many naturally occurring membrane fusion events involve the interaction of calcium with negatively charged phospholipids (e.g., PS and phosphatidylglycerol). Calcium-induced perturbations of membranes containing negatively charged lipids, and the subsequent membrane fusion events, are important mechanisms in many natural membrane fusion processes. Therefore, cochleates can be envisioned as membrane fusion intermediates.

Phase/fluorescent and fluorescent images of Rhodamine-labeled cochleates incubated with splenocytes, indicate that a fusion event occurs between the outer layer of the cochleate and the cell membrane, resulting in the delivery of encochleated material into the cytoplasm of the target cell. As the calcium rich, highly ordered membrane of a cochleate first comes into close approximation to a natural membrane, a perturbation and reordering of the cell membrane is induced, resulting in a fusion event between the outer layer of the cochleate and the cell membrane. This fusion results in the delivery of a small amount of the encochleated material into the cytoplasm of the target cell. The cochleate can then break free of the cell and be available for another fusion event, either with the same or another cell.

Additionally or alternatively, particularly with active phagocytic cells, cochleates may be taken up by endocytosis and fuse from within the endocytic vesicle. Cochleates made with trace amounts of fluorescent lipids have been shown to bind and gradually transfer lipids to the plasma membrane and interior membranes of white blood cells in vitro.

Cochleates are useful for the delivery of bioactive compounds to cultured cells, tissues or organisms by a variety of administration routes. For example, the use of cochleates to deliver protein or peptide molecules as vaccines nas been disclosed in U.S. Pat. No. 5,840,707, issued Nov. 24, 1998. Similarly, polypeptide-cochleates are effective immunogens when administered to animals by intraperitoneal and intramuscular routes of immunization (G. Goodman-Snitkoff, et al., J. Immunol., Vol. 147, p. 410 (1991); M. D. Miller, et al., J. Exp. Med., Vol. 176, p. 1739 (1992)). Further, cochleates are effective delivery vehicles for encapsulated proteins and/or DNA to animals and to cells in culture. For example, reconstituted Sendai or influenza virus glycoproteins are efficiently delivered in encochleated form (Mannino and Gould-Fogerite, Biotechniques 6(1):682-90 (1988); Gould-Fogerite et al., Gene 84:429 (1989); Miller et al., J. Exp. Med. 176:1739 (1992)).

The cochleates can be coadministered with a further agent. The second agent can be delivered in the same cochleate preparation, in a separate cochleate preparation mixed with the cochleate preparation of the invention, separately in another form (e.g., capsules or pills), or in a carrier with the cochleate preparation. The cochleates can further includes one or more additional cargo moieties, such as other drugs, peptides, nucleotides (e.g., DNA and RNA), antigens, nutrients, flavors and/or proteins. Such molecules have been described in U.S. Pat. No. 6,153,217 (Jin et al.) and U.S. Pat. No. 5,994,318 (Gould-Fogerite et al.), and International Patent Publication Nos. WO 96/25942 (Mannino et al.), WO 00/42989 (Zarif et al.) and WO 01/52817 (Zarif et al.). These patents are expressly incorporated by this reference. In some embodiments, the coadministration of the cochleates of the present invention with a further agent provides an additive or synergistic effect, i.e., the coadministration of an apoprotein cochleate and additional agent may provide additional or unexpected therapeutic benefit than each therapy would separately.

One advantage of the cochleates of the present invention is the safety and stability of the composition. Cochleates can be administered orally or by instillation without concern, as well as by the more traditional routes, such as oral, intranasal, intraoculate, intraanal, intravaginal, intrapulmonary, topical, subcutaneous, intradermal, intramuscular, intravenous, subcutaneous, transdermal, systemic, intrathecal (into CSF), and the like. Direct application to mucosal surfaces is an attractive delivery means made possible with cochleates.

Another advantage of the present invention is the ability to modulate cochleate size. Modulation of the size of cochleates and cochleate compositions changes the manner in which the cargo moiety is taken up by cells. For example, in general, small cochleates are taken up quickly and efficiently into cells, whereas larger cochleates are taken up more slowly, but tend to retain efficacy for a longer period of time. Also, in some cases small cochleates are more effective than large cochleates in certain cells, while in other cells large cochleates are more effective than small cochleates.

Cochleates and cochleate compositions can be administered to humans and non-human animals, such as dogs, cats, and farm animals, in food or beverage preparations. Such compositions can be introduced to the food or beverage compositions by the manufacturer (e.g., to supplement food with nutrients), or by the consumer (e.g., where the cochleate composition is sold separately as a food additive). For example, nutrients and/or flavorings may be incorporated into dog or cat food, particularly where such nutrient and/or flavoring is fragile and decomposes or loses activity when exposed to oxygen and/or water.

Cochleates readily can be prepared from safe, simple, well-defined, naturally occurring substances, e.g., PS and calcium. Phosphatidylserine is a natural component of all biological membranes, and is most concentrated in the brain. The phospholipids used can be produced synthetically, or prepared from natural sources. Soy PS is inexpensive, available in large quantities and suitable for use in humans. Additionally, clinical studies indicate that PS is safe and may play a role in the support of mental functions in the aging brain. Unlike many cationic lipids, cochleates (which are composed of anionic lipids) are non-inflammatory and biodegradable. The tolerance in vivo of mice to multiple administrations of cochleates by various routes, including intravenous, intraperitoneal, intranasal and oral, has been evaluated. Multiple administrations of high doses of cochleate formulations to the same animal show no toxicity, and do not result in either the development of an immune response to the cochleate matrix, or any side effects relating to the cochleate vehicle.

Accordingly, in yet another aspect, the present invention provides for both prophylactic and therapeutic methods of treating a subject at risk of (or susceptible to) a disorder or having a disorder which can be treated with one or more apoprotein or plasmid encoding one or more apoprotein.

In one embodiment, the present invention provides a method for treating a disease or disorder in a subject that includes the step of administering to said subject a therapeutically effective amount of a cochleate composition that includes a cochleate including a negatively charged lipid component and a multivalent cation component, and an apoprotein component associated with the cochleate. The diseases or disorders treated by the method of the invention can include but are not limited to atherosclerosis, hypertension, hyperlipidemia, hypercholesterolemia, Alzheimer's disease, coronary heart disease and/or cardiovascular disease. In some embodiments, the cochleates of the present invention may also be useful in the treatment of thrombosis or thrombotic disorders, e.g., deep vein thrombosis (DVT), unstable angina, myocardial infarction, acute ischaemic stroke, and stroke. Thrombosis and thrombotic disorders are those associated with the existence or development of a blood clot inside a blood vessel or cavity of the heart.

The apoproprotein component can include any of the apoproteins described herein, including but not limited to one or more apolipoproteins, complexed or uncomplexed.

The apoprotein can be associated with the cochleate in any of the methods described herein. For example, in one embodiment, the apoprotein component is associated with the cochleate, such that the apoprotein component dissociates with the cochleate upon contact with a target environment. The apoprotein can be bound to a component of the cochleate with any of the linkers described herein, e.g., a linker that is reducible, or otherwise reversible or digestible by an enzyme, protein, or molecule endogenous to the target environment. The enzyme can be an extracellular, intracellular or endosomal enzyme endogenous to the subject. In another embodiment, the apoprotein component is electrostatically associated with the cochleate and dissociates with the cochleate upon contact with a pH gradient in a cell or organ of the subject.

In a particularly preferred embodiment, the cochleate composition induces reverse cholesterol transport in one or more macrophages. When employed to do so, the apoprotein-cochleates of the instant invention can be used to treat atherosclerosis.

The methods of the present invention include methods of administering a apoprotein to a host, wherein the apoprotein is incorporated into a cochleate or cochleate composition of the invention. The administration route can be mucosal, systemic, oral, nasal, topical, intranasal, intraocular, intrarectal, intravaginal, intrapulmonary, intravenous, intramuscular, subcutaneous, transdermal and intradermal, buccal, sublingual, rectal, vaginal or parenteral.

The present invention provides a method for treating a subject that would therapeutic indication that would benefit from an apoprotein can be treated by the methods of the invention. The method includes the step of administering to the subject a composition of the invention, such that the disease or disorder is treated. In some embodiments, the cochleates of the present invention may be administered solely to supplement levels of apoprotein in a subject. In yet other embodiments, the cochleates of the present invention are administered to deliver a mutant form of an apoprotien, e.g., apoA-IM, which may have a greater effect than the endogenous form.

The above methods can be employed in the absence of other treatment, or in combination with other treatments. Such treatments can be started prior to, concurrent with, or after the administration of the compositions of the instant invention. Accordingly, the methods of the invention can further include the step of administering a second treatment, such as a second treatment for the disease or disorder or to ameliorate side effects of other treatments. Such second treatment can include, e.g., high blood pressure medication and any treatment directed toward treating hypertension or atherosclerosis. Additionally or alternatively, further treatment can include administration of drugs to further treat the disease or to treat a side effect of the disease or other treatments (e.g., anti-nausea drugs).

In one aspect, the invention provides a method for preventing in a subject, a disease or disorder which can be treated with administration of the compositions of the invention. Subjects at risk for a disease or condition which can be treated with the agents mentioned herein can be identified by, for example, any or a combination of diagnostic or prognostic assays known to those skilled in the art. Administration of a prophylactic agent can occur prior to the manifestation of symptoms characteristic of the disease or disorder, such that the disease or disorder is prevented or, alternatively, delayed in its progression.

Another aspect of the invention pertains to methods of administering a cochleate composition for therapeutic purposes. In one embodiment, the present invention provides a method for treating a subject that would benefit from administration of a composition of the present invention. Any therapeutic indication that would benefit from a cochleate composition of the invention can be treated by the methods of the invention. The present invention provides methods of treating a subject at risk for or having a disease or disorder which can be treated with one or more cargo moiety. The method includes the step of administerating to the subject a composition of the invention, such that the disease or disorder is prevented, ameliorated, terminated or delayed in its progression. The disease or disorder can be any of the diseases or disorders discussed herein.

In yet another aspect, the invention pertains to uses of the cochleate compositions of the invention for prophylactic and therapeutic treatments as described infra. Accordingly, the compounds of the present invention can be incorporated into pharmaceutical compositions suitable for administration. Such compositions typically comprise the compositions of the invention and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Wetting agents, emulsifiers and lubricants, such as sodium lauryl sulfate and magnesium stearate, as well as coloring agents, release agents, sweetening, flavoring and perfuming agents, preservatives and antioxidants may also be present in the compositions.

Examples of pharmaceutically acceptable antioxidants, which may also be present in formulations of therapeutic compounds of the invention, include water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Furthermore, the present invention can further include one or more additional agents, including water, antimicrobial agents, pasticizing agents, flavoring agents, surfactants, stabilizing agents, emulsifying agents, thickening agents, binding agents, coloring agents, sweeteners, fragrances, and the like.

Suitable antimicrobial agents include parabens, triclosan, cetyl pyridium chloride, domiphen bromide, quaternary ammonium salts, zinc compounds, sanguinarine, fluorides, alexidine, octonidine, EDTA, and essential oils such as thymol, methyl salicylate, menthol and eucalyptol.

Suitable plasticizing agents include, for example, polyols such as sugars, sugar alcohols, or polyethylene glycols (PEGs), urea, glycol, propylene glycol, triethyl citrate, dibutyl or dimethyl phthalate, monoacetin, diacetin or triacetin.

Suitable surfactants include pluronic acid, sodium lauryl sulfate, mono and diglycerides of fatty acids and polyoxyethylene sorbitol esters, such as, Atmos 300 and Polysorbate 80. Suitable stabilizing agents include xanthan gum, locust bean gum, guar gum, and carrageenan. Suitable emulsifying agents include triethanolamine stearate, quaternary ammonium compounds, acacia, gelatin, lecithin, bentonite, veegum, and the like. Suitable thickening agents include methylcellulose, carboxyl methylcellulose, and the like. Suitable binding agents include starch.

Suitable sweeteners that can be included are those well known in the art, including both natural and artificial sweeteners. Suitable sweeteners include water-soluble sweetening agents such as monosaccharides, disaccharides and polysaccharides; water-soluble artificial sweeteners such as soluble saccharin salts, cyclamate salts, or the free acid form of saccharin, and the like; dipeptide based sweeteners, such as L-aspartic acid derived sweeteners; water-soluble sweeteners derived from naturally occurring water-soluble sweeteners, such as a chlorinated derivative of ordinary sugar (sucrose), known, under the product description of sucralose; and protein based sweeteners such as thaumatoccous danielli (Thaumatin I and II).

In general, an effective amount of auxiliary sweetener is utilized to provide the level of sweetness desired for a particular composition, and this amount will vary with the sweetener selected. This amount will normally be 0.01% to about 10% by weight of the composition when using an easily extractable sweetener.

The flavorings that can be used include those known to the skilled artisan, such as natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, and fruit essences. These flavorings can be used individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used.

The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation.

The compositions of this invention can also contain coloring agents or colorants. The coloring agents are used in amounts effective to produce the desired color. The coloring agents useful in the present invention include pigments such as titanium dioxide, which may be incorporated in amounts of up to about 5 wt %, and preferably less than about 1 wt %. Colorants can also include natural food colors and dyes suitable for food, drug and cosmetic applications. These colorants are known as FD&C dyes and lakes. A full recitation of all FD&C and D&C dyes and their corresponding chemical structures may be found in the Kirk-Othmer Encyclopedia of Chemical Technology, Volume 5, Pages 857-884, which text is accordingly incorporated herein by reference.

Formulations of the present invention include those suitable for oral, nasal, topical, transdermal, buccal, sublingual, rectal, vaginal or parenteral administration. The formulations may conveniently be presented in unit dosage form and may be prepared by any methods well known in the art of pharmacy. The amount of active ingredient which may be combined with a carrier material to produce a single dosage form will generally be that amount of the composition which produces a therapeutic effect.

Methods of preparing the compositions of the invention may include the step of bringing into association a composition of the present invention with a carrier and, optionally, one or more accessory ingredients. In general, the formulations are prepared by uniformly and intimately bringing into association a composition of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) or as mouth washes and the like, each containing a predetermined amount of a composition of the present invention as an active ingredient. A composition of the present invention may also be administered as a bolus, electuary, or paste.

In solid dosage forms of the invention for oral administration (capsules, tablets, pills, dragees, powders, granules, and the like), the active ingredient (e.g., dried or lyophilized cochleates of the invention) is mixed with one or more pharmaceutically acceptable carriers, such as sodium citrate or dicalcium phosphate, or any of the following: fillers or extenders, such as starches, lactose, sucrose, glucose, mannitol, or silicic acid; binders, such as, for example, carboxymethylcellulose, alginates, gelatin, polyvinyl pyrrolidone, sucrose or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents.

In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatom or hydroxyproplemethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered composition moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes or microspheres.

The composition may be sterilized by, for example, filtration through a bacteria-retaining filter, or by incorporating sterilizing agents in the form of sterile solid compositions which may be dissolved in sterile water, or some other sterile injectable medium immediately before use.

These compositions may also optionally contain opacifying agents and may be of a composition that they release the active ingredient only, or preferentially, in a certain portion of the gastrointestinal tract, optionally, in a delayed manner. Examples of embedding compositions which may be used include polymeric substances and waxes. The active ingredient may also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert dilutents, the oral compositions may also include adjuvants such as wetting agents, emulsifying and suspending agents, sweetening, flavoring, coloring, perfuming and preservative agents.

Suspension, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a composition of this invention include powders, sprays, ointments, pastes, creams, lotions, gels, solutions, patches and inhalants. The composition may be mixed under sterile conditions with a pharmaceutically acceptable carrier, and with any preservatives, buffers, or propellants which may be required.

The ointments, pastes, creams and gels may contain, in addition to an composition of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a composition of the present invention to the body. Such dosage forms may be made by dissolving or dispersing the composition in the proper medium. Absorption enhancers may also be used to increase the flux of the composition across the skin. The rate of such flux may be controlled by either providing a rate controlling membrane or dispersing the composition in a polymer matrix or gel.

Pharmaceutical compositions of this invention suitable for parenteral administration comprise a cochleate or cochleate composition of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifingal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride, in the composition. Prolonged absorption of the injectable compositions can be brought about by including in the composition an agent which delays absorption, for example, aluminum monostearate and gelatin.

Sterile injectable solutions can be prepared by incorporating a composition of the invention in the desired amount in an appropriate solvent with one or a combination of ingredients enumerated above, as required, followed by filtered sterilization. Generally, dispersions are prepared by incorporating the composition into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the cochleate compositions of the invention plus any additional desired ingredient from a previously sterile-filtered solution thereof.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions which are compatible with body tissue.

The term “therapeutically effective amount” refers to the amount necessary or sufficient to produce a desired physiologic response. The effective amount may vary depending on such factors as the size and weight of the subject, or the particular compound. The effective amount may be determined through consideration of the toxicity and therapeutic efficacy of the compounds by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it may be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to unaffected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the EC50 (i.e., the concentration of the test composition that achieves a half-maximal response) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

The pharmaceutical compositions can be included in a container along with one or more additional compounds or compositions and instructions for use. For example, the invention also provides for packaged pharmaceutical products containing two agents, each of which exerts a therapeutic effect when administered to a subject in need thereof. Alternatively, the invention provides for packaged pharmaceutical products containing two agents, the first of which exerts a therapeutic effect and the second of which, e.g., acts as a carrier. For example, the present invention may provide a dried or lyophilized cochleate and a buffer for administration. Alternately, the present invention may provide a concentrated solution of cochleates and a buffer for dilution prior to administration. A pharmaceutical composition may also comprise a third agent, or even more agents yet, wherein the third (and fourth, etc.) agent can be another agent against the disorder, such as a hypertension treatment. In some cases, the individual agents may be packaged in separate containers for sale or delivery to the consumer. The agents of the invention may be supplied in a solution with an appropriate solvent or in a solvent-free form (e.g., lyophilized). Additional components may include acids, bases, buffering agents, inorganic salts, solvents, antioxidants, preservatives, and the like. The additional kit components are present as pure compositions, or as aqueous or organic solutions that incorporate one or more additional kit components. Any or all of the kit components optionally further comprise buffers.

This invention is further illustrated by the following examples which should not be construed as limiting.

### EXAMPLE

### Preparation of apoA-I Cochleates via the DC and LC Methods

2.00 mg human apoA-I from plasma (Sigma, 1.31 mg/mL in NH₃HCO₃, pH 7.4) is thawed prior to use and diluted with an equal volume of extraction buffer (2M NaCl, 0.2M NaPO₄, pH 7.4). 60 mg octylglucoside is added to the mixture, followed by 8 mg of DOPS, and the mixture is vortexed. The mixture is then allowed to incubate at 37° C. for 45 minutes, followed by filter sterilization. The mixture is then divided into two equal parts, Solution A and Solution B, each approximately 1.5 mL.

A. DC Cochleates.

Solution A was dialyzed at room temperature overnight against 250 ML TES buffer (2 mM TES N-Tris[hydroxymethyl]-methyl-2 aminoethane sulfonic acid, 2 mM L-histidine, 100 mM NaCl, pH 7.4) containing 3 mM CaCl₂. Solution A was further dialyzed against four additional changes of 250 mL TES buffer with 3 mMCaCl₂. From a final volume of 1.85 mL, white calcium-phospholipid precipitates were isolated.

B. LC Cochleates.

Solution B first was dialyzed overnight using 250 mL TES buffer without divalent cations, followed by two additional changes of buffer. The resulting vesicles were converted to a cochleate precipitate by dialysis against two changes of TES buffer containing 3 mM Ca²⁺ ions.

We claim:

1. An apoprotein-cochleate composition comprising:
a cochleate comprising a negatively charged lipid component and a multivalent cation component; and
an apoprotein component associated with the cochleate.

2. The composition of claim 1, wherein the apoprotein component comprises at least one apolipoprotein.

3. The composition of claim 1, wherein the apoprotein component comprises at least one apolipoprotein selected from the group consisting of apolipoprotein A (apoA-I), apolipoprotein E (Apo-E), apolipoprotein-Milano (apoA-IM), apolipoprotein-Milano dimers, proapolipoprotein (proapo AI), recombinant apoA-I, apolipoprotein B, apolipoprotein A-II, apolipoprotein A-IV, apolipoprotein C-I, apolipoprotein Lp(a), apolipoprotein alleles, and apolipoprotein D.

4. The composition of claim 1, further comprising an anti inflammatory drug.

5. The composition of claim 1, wherein the apoprotein component comprises an apoprotein complexed with a phospholipid.

6. The composition of claim 1, wherein the apoprotein component comprises an apoprotein bound to a cochleate component.

7. The composition of claim 6, wherein the apoprotein component is associated with the cochleate, such that the apoprotein component dissociates with the cochleate upon contact with a target environment

8. The composition of claim 6, wherein the apoprotein is bound by an electrostatic, hydrophobic, covalent, or ionic interaction with the cochleate component.

9. The composition of claim 8, wherein the apoprotein is electrostatically associated with the cochleate and dissociates with the cochleate upon contact with a pH gradient in a cell or organ of the subject.

10. The composition of claim 6, wherein the apoprotein is bound to the cochleate component with a digestible, reducible, or otherwise reversible linker.

11. The composition of claim 10, wherein digestible, reducible, or otherwise reversible linker is digested, reduced or otherwise reduced by an enzyme, protein or molecule endogenous to the target environment.

12. The composition of claim 11, wherein the enzyme is an extracellular, intracellular, or endosomal enzyme endogenous to the subject.

13. The composition of claim 1 further comprising a pharmaceutically acceptable carrier.

14. The composition of claim 13, wherein the composition is in the form of a pill, capsule, lozenge, or liquid for oral administration.

15. A method for preparing an apoprotein cochleate of claim 1 comprising:
introducing an apoprotein to a liposomal suspension or lipid solution; and
contacting the resulting suspension or solution with a multivalent cation to form an apoprotein cochleate.

16. A method for treating a disease or disorder in a subject comprising:
administering to said subject a therapeutically effective amount of a cochleate composition comprising:
a cochleate comprising a negatively charged lipid component and a multivalent cation component, and
an apoprotein component associated with the cochleate.

17. The method of claim 16, wherein the disease or disorder is atherosclerosis, hypertension, hyperlipidemia, hypercholesterolemia, Alzheimer's disease, coronary heart disease, or cardiovascular disease.

18. The method of claim 16, wherein the treatment reduces the level of artherosclerotic plaque in the subject, reduces serum cholesterol levels in the subject, increases the exflux of cholesterol from macrophages in a subject, or lessens or alleviates hypercholesterolemia.

19. The method of claim 16, wherein the administration route is selected from the group consisting of mucosal, systemic, oral, intranasal, intraocular, intrarectal, intravaginal, intrapulmonary, intravenous, intramuscular, subcutaneous, transdermal and intradermal.

20. The method of claim 16, wherein the cochleate composition induces reverse cholesterol transport in one or more macrophages.

21. A cochleate composition comprising:
a cochleate comprising a negatively charged lipid component and a multivalent cation component; and
a plasmid that encodes for an apoprotein associated with the cochleate.

22. The composition of claim 21, wherein the plasmid encodes at least one apolipoprotein.

23. The composition of claim 21, wherein the plasmid encodes at least one apolipoprotein selected from the group consisting of apolipoprotein A (apoA-I), apolipoprotein E (apo-E), apolipoprotein-Milano (apoA-IM), apolipoprotein-Milano dimers, proapolipoprotein (proapo AI), recombinant apoA-I, apolipoprotein B, apolipoprotein A-II, apolipoprotein A-IV, apolipoprotein C-I, apolipoprotein Lp(a), apolipoprotein alleles, and apolipoprotein D.

24. The compositions of claim 21, further comprising an apoprotein.

25. The composition of claim 21, further comprising an anti inflammatory drug.
